(19) United States
(12) Patent Application Publication
(10) Pub. No.: US 2013/0022545 A1
(43) Pub. Date: Jan. 24, 2013

(54) DRUG DELIVERY SYSTEM FOR TREATMENT OF LIVER CANCER BASED ON INTERVENTIONAL INJECTION OF TEMPERATURE AND pH-SENSITIVE HYDROGEL

(71) Applicants: Doo Sung Lee, Gwacheon-si (KR); Bong Sup Kim, Suwon-si (KR); Cong Truc Huynh, Suwon-si (KR)

(72) Inventors: Doo Sung Lee, Gwacheon-si (KR); Bong Sup Kim, Suwon-si (KR); Cong Truc Huynh, Suwon-si (KR)

(73) Assignee: Research & Business Foundation Sungkyunkwan University, Suwon-si (KR)

(21) Appl. No.: 13/551,889

(22) Filed: Jul. 18, 2012

(30) Foreign Application Priority Data
Jul. 18, 2011 (KR) 10-2011-0071184

(51) Int. Cl. A61K 31/785; A61K 31/795; A61P 35/00; A61K 49/00

(52) U.S. Cl. 424/9.1; 424/78.17

(57) ABSTRACT

A drug delivery system for the treatment of liver cancer that is based on interventional injection of a temperature and pH-sensitive hydrogel is provided. The drug delivery system is composed of a block copolymer applicable to hepatic arterial catheterization and a therapeutic agent is loaded inside the drug delivery system, and the drug delivery system is in the sol state outside, and undergoes a phase transition into the gel state inside the hepatic artery, thereby delaying or blocking blood supply of the hepatic artery, and slowly releasing the therapeutic agent during the phase transition into the gel state inside the hepatic artery.

### CROSS-REFERENCE TO RELATED APPLICATIONS

This application claims the benefit under 35 U.S.C. §119(a) of Korean Patent Application No. 10-2011-0071184 filed on Jul. 18, 2011, in the Korean Intellectual Property Office, the entire disclosure of which is incorporated herein by reference for all purposes.

### BACKGROUND

1. Field

The following description relates to a drug delivery system for the treatment of liver cancer, which is based on interventional injection of temperature and pH-sensitive hydrogel.

2. Description of Related Art

Liver cancer is a malignant tumor carrying a very poor prognosis, and is the second leading cause of cancer death in Korea, and the third leading cause of cancer death worldwide. Unfortunately, more than 70% of liver cancer patients cannot undergo radical surgery, and more than 50% of patients had a recurrence at other sites within 5 years, even after radical surgery. Response rates of the advanced liver cancer to systemic chemotherapy are as low as 10%. Current treatment options for liver cancer are intra-arterial chemotherapy and transcatheter arterial chemoembolization (TACE). TACE is the most frequently performed procedure in the treatment of unresectable liver tumors. In this procedure, the blood supply to a tumor is blocked after anticancer drugs are given in the hepatic artery that supplies nutrients to the hepatoma. In some cases, anticancer drugs are only given without blocking off the blood vessels. Lipiodol (Laboratorie Gurerbet, Aulnay-Sous-Bios, France, iodine content: 38 wt %) is an embolic material used in TACE, and it is selectively accumulated and stays longer in the neovessels of liver cancer tissues, and thus is able to deliver higher concentrations of anticancer drugs to hepatomas than to normal tissues.

Liver tissue is known to receive oxygen from the aorta via the hepatic artery and nutrients from the small and large intestines via the portal vein. Normal liver tissue receives 70% or more of its blood supply from the portal vein, while liver cancer tissue receives 90% or more of its blood supply from the hepatic artery. Therefore, embolization of the hepatic artery after hepatic arterial administration of anticancer drugs can lead to selective necrosis of the liver tumor while it leaves normal tissues virtually unaffected. In this method, various anticancer drugs such as doxorubicin (DOX), paclitaxel, docetaxel, cisplatin, and carboplatin are used by dispersing them in a contrast agent such as the embolic material Lipiodol. This system including Lipiodol and the above-mentioned anticancer drugs, however, is physically unstable and therefore has many limitations during the operation.

Korean Patent No. 10-0539451 discloses a paclitaxel composition used for transcatheter arterial chemoembolization by solubilizing paclitaxel in an oily contrast agent, and the preparation method thereof. This technique relates to an oily paclitaxel composition additionally including chemicals that prevent paclitaxel precipitation, and thus has an advantage of delivering anticancer drug to the target cells by chemoembolization since it is possible to visualize the blood vessel during the chemoembolization process.

Korean Patent No. 10-0497258 relates to a paclitaxel mixed composition for chemoembolization and its water-in-oil type emulsion formulation, and discloses a paclitaxel mixed composition that can be formulated into an emulsion applicable to chemoembolization, its water-in-oil (w/o) type emulsion formulation, and a preparation method thereof, in which the mixed composition is used to prepare an emulsion formulation for chemoembolization including an iodized oily contrast medium and an aqueous contrast medium.

In addition, anticancer agents such as adrimicin and epirubicin are used conventionally for the treatment of hepatoma in radiology. These anticancer agents are water-soluble materials, so it is impossible to directly dissolve them in Lipiodol, and thus suspension type formulations have been used. However, these suspension type formulations cannot be stored for a prolonged period of time because of aggregation and precipitation of particles.

Meanwhile, currently available anticancer drugs have many problems in terms of toxicity and side effects, and thus many studies have been made to develop anticancer/chemotherapeutic agents having low toxicity and side effects and excellent efficacy.

The ideal goals for anticancer chemotherapy are to directly deliver the anticancer materials to tumors with a higher concentration than to normal tissues and to find anticancer materials that are selectively active on tumor cells with minimal toxicity against normal cells.

### SUMMARY

In one general aspect, there is provided a drug delivery system for the treatment of liver cancer that is based on interventional injection of a temperature and pH-sensitive hydrogel, where the drug delivery system is composed of a block copolymer applicable to hepatic arterial catheterization and a therapeutic agent is loaded inside the drug delivery system, and the drug delivery system is in the sol state outside, and undergoes a phase transition into the gel state inside the hepatic artery, thereby delaying or blocking blood supply of the hepatic artery, and slowly releasing the therapeutic agent during the phase transition into the gel state inside the hepatic artery.

The drug delivery system may further include that a contrast agent is further included inside the drug delivery system.

The drug delivery system may further include that the block copolymer is a temperature and pH-sensitive poly(β-aminoester)-based block copolymer, a poly(amidoamine)-based block copolymer, a poly(aminoesterurethane)-based block copolymer, a poly(aminourethaneurea)-based block copolymer, or a sulfonamide-based block copolymer.

The drug delivery system may further include that a hydrophilic block component of the block copolymer is polyethylene glycol.

The drug delivery system may further include that a hydrophobic and biodegradable block component of the block copolymer is one or more selected from the group consisting of polylactic acid (PLA), polyglycolic acid (PGA), polycaprolactone (PCL), poly(caprolactone-lactide) random copolymer (PCLA), poly(caprolactone-glycolide) random copolymer (PCGA), and poly(lactide-glycolide) random copolymer (PLGA).

The drug delivery system may further include that the poly(β-amino ester)(PAE)-based block copolymer is poly(β-amino ester)-polyethylene glycol-poly(β-amino ester) (PAE-PEG-PAE), PAE-PCLA-PEG-PCLA-PAE, PAE-PCL-PEG-PCL-PAE, or PAE-PCGA-PEG-PCGA-PAE.

The drug delivery system may further include that the poly(β-amino ester)-based block copolymer is defined by the following Chemical Formula:

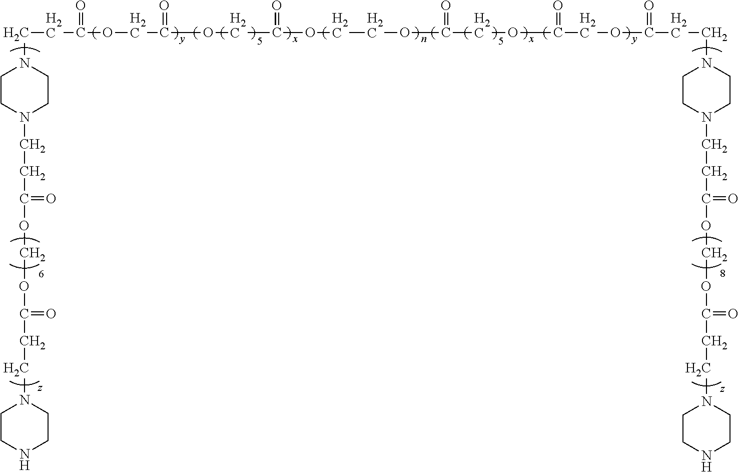

where x and y are independently an integer ranging from 0 to 50, and z and n are independently an integer ranging from 1 to 100.

The drug delivery system may further include that the poly(amidoamine)(PAA)-based block copolymer is a poly(amidoamine)-polyethylene glycol-poly(amidoamine) (PAA-PEG-PAA), PAA-PCLA-PEG-PCLA-PAA, PAA-PCL-PEG-PCL-PAA, or PAA-PCGA-PEG-PCGA-PAA copolymer.

The drug delivery system may further include that the poly(amidoamine)-based block copolymer is defined by the following Chemical Formula:

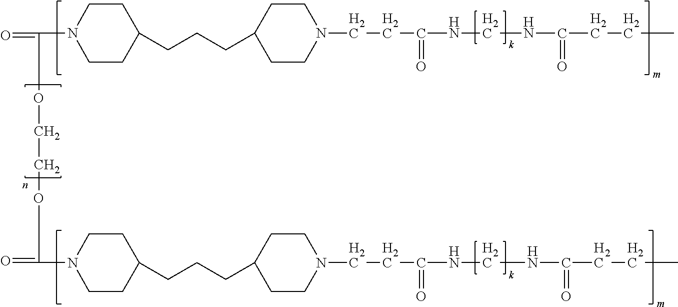

where k is an integer ranging from 4 to 10, n is an integer ranging from 11 to 45, and m is an integer ranging from 10 to 100.

The drug delivery system may further include that the poly(aminoester urethane) (PAEU)-based block copolymer is polyamino ester urethane-polyethylene glycol-polyamino ester urethane (PAEU-PEG-PAEU), PAEU-PCLA-PEG-PCLA-PAEU, PAEU-PCL-PEG-PCL-PAEU, PAEU-PCGA-PEG-PCGA-PAEU copolymer or (PCLA-PEG-PCLA-PAEU)x multicopolymer (wherein x is the number of repeating unit ranging from 1 to 20), 4-arm PEG-PAEU copolymer.

The drug delivery system may further include that the poly(amino ester urethane)-based block copolymer is defined by the following Chemical Formula:

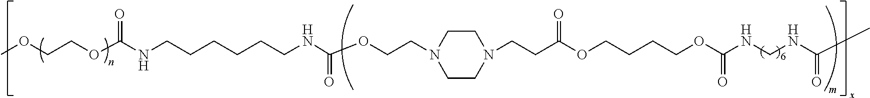

where n and m are independently the number of repeating unit ranging from 2 to 10, and x is the number of repeating unit ranging from 1 to 20.

The drug delivery system may further include that the poly(aminourethaneurea) (polyβ-aminourethaneurea), PAUU)-based block copolymer is polyaminourethaneurea-polyethyleneglycol-polyaminourethaneurea (PAUU-PEG-PAUU), PAUU-PCLA-PEG-PCLA-PAUU, PAUU-PCL-PEG-PCL-PAUU, PAUU-PCGA-PEG-PCGA-PAUU block copolymer, (PCLA-PEG-PCLA-PAUU)x multiblock copolymer, (PEG-PAUU)x multiblock copolymer (wherein x is the number of repeating unit ranging from 1 to 20), or 4-arm PEG-PAUU block copolymer.

The drug delivery system may further include that the poly(aminourethaneurea)-based multiblock copolymer is defined by the following Chemical Formula:

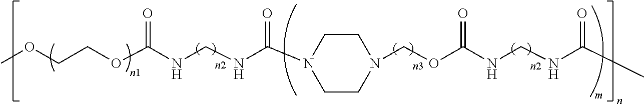

where n1 is an integer ranging from 7 to 50, n2 is an integer ranging from 2 to 8, n3 is an integer ranging from 1 to 10, and m is an integer ranging from 2 to 6.

The drug delivery system may further include that the sulfonamide(SAM)-based block copolymer is a sulfonamide-polyethylene glycol-sulfonamide (SAM-PEG-SAM), SAM-PCLA-PEG-PCLA-SAM, SAM-PCL-PEG-PCL-SAM or SAM-PCGA-PEG-PCGA-SAM copolymer.

The drug delivery system may further include that the sulfonamide-based block copolymer is defined by the following Chemical Formula:

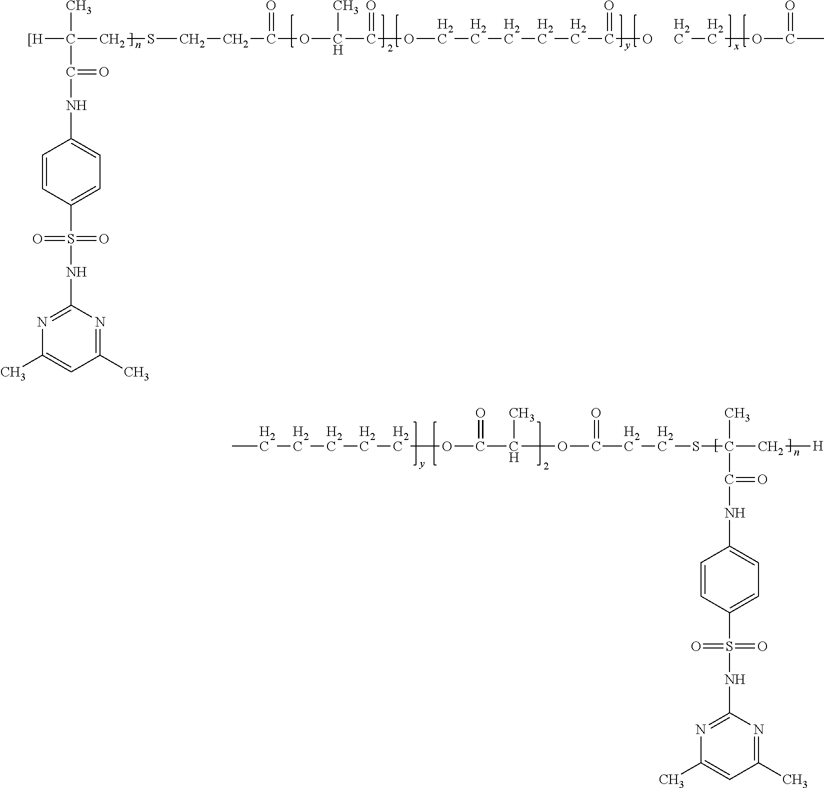

where x is an integer ranging from 10 to 50, y and z are independently an integer ranging from 0 to 50, and z and n are independently the number of repeating unit ranging from 1 to 100.

The drug delivery system may further include that the contrast agent is included in a volume of 5 to 20 volume%, based on the volume of the copolymer.

The drug delivery system may further include that an initial release coefficient is 0.5 to 0 when the initial release coefficient is defined by the slope of the curve at the point of origin in the time-release curve of the drug delivery system.

Other features and aspects may be apparent from the following detailed description, the drawings, and the claims.

### BRIEF DESCRIPTION OF THE DRAWINGS

FIG. 1 is an X-ray image of the anticancer drug-encapsulated temperature and pH-sensitive block copolymer/doxorubicin/Lipiodol formulation of Example 11 that was injected into the rabbit hepatoma via a hepatic artery guiding catheter.

FIG. 2 is a CT image of the anticancer drug-encapsulated temperature and pH-sensitive block copolymer/doxorubicin/Lipiodol formulation of Example 11 that was injected into the rabbit hepatoma via a hepatic artery guiding catheter.

FIG. 3 is a graph showing the concentrations of doxorubicin that was released from the hepatoma and normal tissue during 2 weeks after injection of the anticancer drug-encapsulated temperature and pH-sensitive block copolymer/doxorubicin(6 mg)/Lipiodol formulation of Examples 16 to 18 into the rabbit hepatoma via a hepatic artery guiding catheter.

FIG. 4 is a photograph of the liver tissue that was excised after interventional injection according to Example 16.

FIG. 5 is a photograph showing the hematoxylin & eosin staining of the liver tissue that was excised after interventional injection according to Example 16.

FIG. 6 is a photograph showing the smooth consistency of VX2 carcinoma of the hepatoma model according to Example 16.

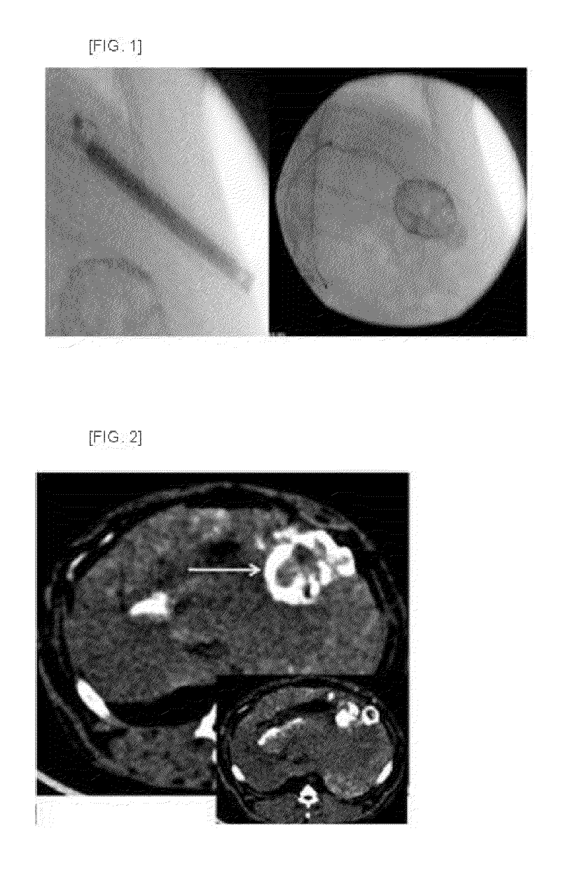

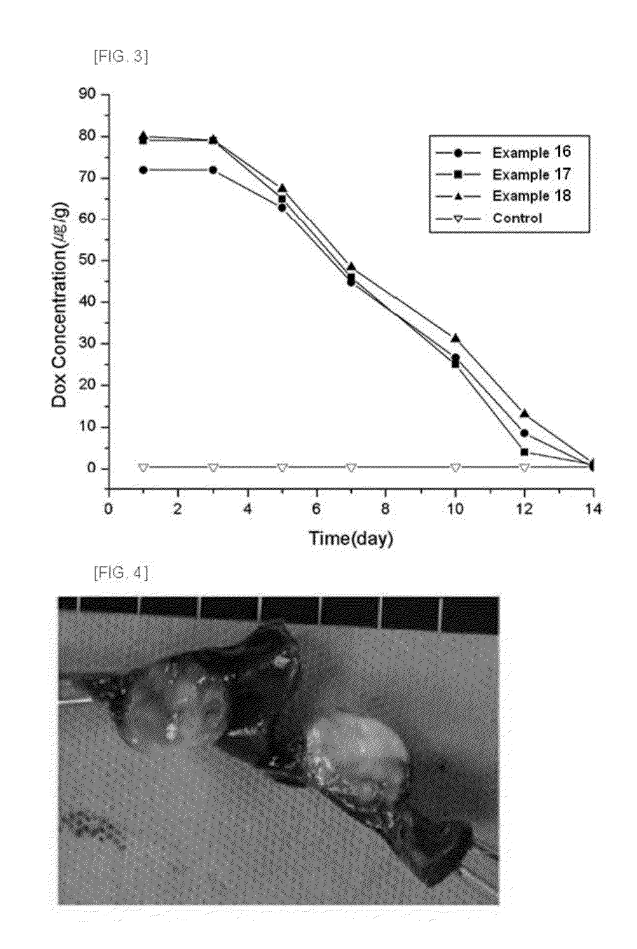

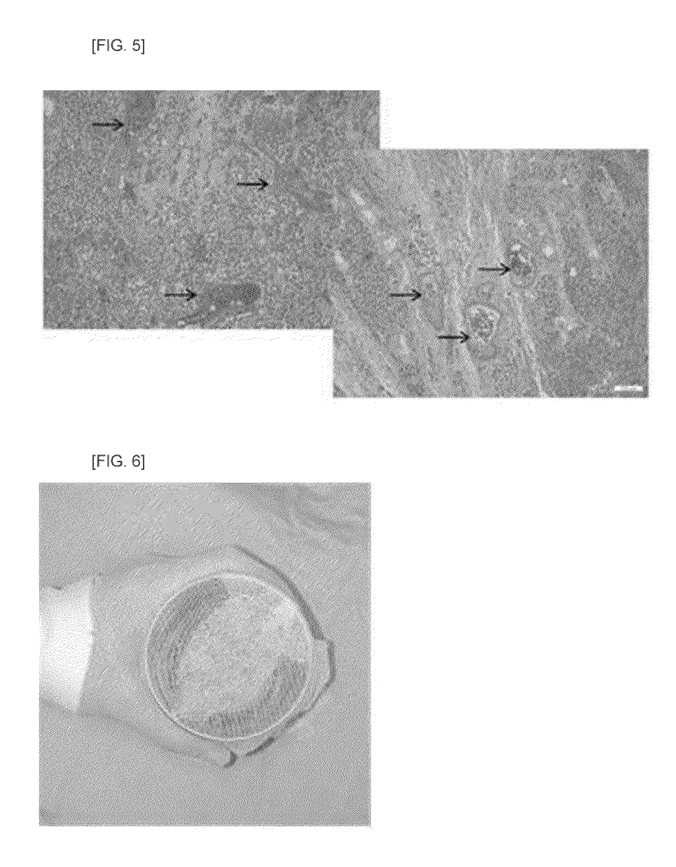

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be suggested to those of ordinary skill in the art. Also, descriptions of well-known functions and constructions may be omitted for increased clarity and conciseness.

Hydrogel is a substance having a swelling property in an aqueous solution, and generally composed of hydrophilic organic polymers to form a cross-linked network structure via covalent or non-covalent bonds, and thus structurally stable. It also has properties of absorbing and releasing a large amount of solvent, and thus has transport properties like in a fluid. Owing to these physical properties, hydrogels are used as an ideal candidate in various fields, such as in vivo diagnostics, drug/gene/cell delivery, chemical separations, chemical and biological sensors, and optical materials.

In the temperature and pH-sensitive polymer hydrogel, a hydrophobic polymer binds to a temperature-sensitive and biodegradable hydrophilic polymer, and pH-sensitive components bind at both ends of the temperature-sensitive polymer. It is in the sol state at room temperature, but undergoes a phase transition into the gel state under the body conditions of pH 7.0 to 7.4 and temperature 37° C. and exists in a polymer hydrogel. That is, it is prepared by various synthetic methods and molecular design in order to induce ionization of pH-sensitive components at pH 7.0 to 7.4

The temperature and pH-sensitive block copolymer may be composed of a pH sensitive poly(β-amino ester)-based block copolymer, a pH sensitive poly(amidoamine)-based copolymer, a pH-sensitive poly(amino ester urethane)-based block copolymer, a pH-sensitive poly(aminourethaneurea)-based multiblock copolymer, or a pH-sensitive sulfonamide-based block copolymer.

This block copolymer sensitive to pH as well as temperature is gelled at pH range of 7.0 to 7.4 similar to pH range in the body, whereas it transforms in a sol-state at the above pH range or less. It was recognized that gel is stably formed in the body without a conventional problem in the temperature-sensitive hydrogels such as the clogging phenomenon of injection needles occurring during injection. Thus, it was suggested that an injectable hydrogel prepared thereby can be used as a drug carrier for sustained-release target delivery system at a specific temperature and pH, leading to use as an anticancer drug carrier for guiding catheterization.

Further, the block copolymer was designed such that its main chain has an ester bond, and thus it is excreted from the body via biodegradation after injection of the hydrogel into the hepatoma. In order to control biodegradation rate in the body, polyethylene glycol (PEG) may be used as the hydrophilic block component of the block copolymer, and one or more selected from the group consisting of polylactic acid (PLA), polyglycolic acid (PGA), polycaprolactone (PCL), poly(caprolactone-lactide) random copolymer (PCLA), poly(caprolactone-glycolide) random copolymer (PCGA), and poly(lactide-glycolide) random copolymer (PLGA) may be used as the hydrophobic and biodegradable block component.

In addition, the pH-sensitive poly(β-amino ester) (PAE)-based block copolymer may be a poly(β-amino ester)-polyethylene glycol-poly(β-amino ester) (PAE-PEG-PAE), PAE-PCLA-PEG-PCLA-PAE, PAE-PCL-PEG-PCL-PAE or PAE-PCGA-PEG-PCGA-PAE copolymer. Specifically, among the pH-sensitive poly(β-amino ester) (PAE)-based block copolymers, the PAE-PCLA-PEG-PCLA-PAE block copolymer may be used in the injectable drug delivery system including the block copolymer represented by the following Chemical Formula 1, which is based on interventional injection of temperature and pH-sensitive block copolymer hydrogel.

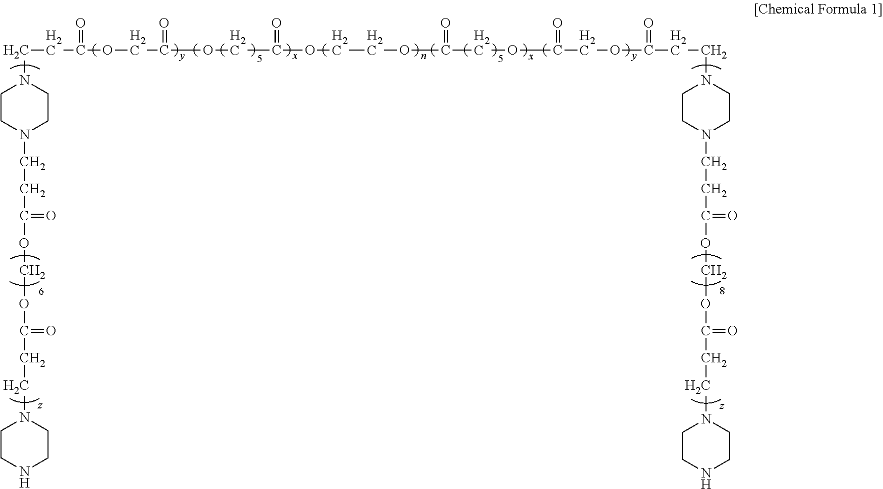

where x and y are independently an integer ranging from 0 to 50, and z and n are independently an integer ranging from 1 to 100.

Further, the hydrophilic PEG block may have a molecular weight of 1,500 to 3,000, and a molar ratio of PEG and PCL may be 1:1.5 to 1:2.5, and a molar ratio of the hydrophobic PCL and PLA blocks may be 2:1 to 3:1, and PAE may have a molecular weight of 1,000 to 1,500. Meanwhile, the pH-sensitive poly(amidoamine) (PAA)-based block copolymer may be a poly(amidoamine)-polyethylene glycol-poly(amidoamine) (PAA-PEG-PAA), PAA-PCLA-PEG-PCLA-PAA, PAA-PCL-PEG-PCL-PAA or PAA-PCGA-PEG-PCGA-PAA copolymer.

Specifically, among the poly(amidoamine) (PAA)-based block copolymers, the PAA-PEG-PAA block copolymer may be a block copolymer represented by the following Chemical Formula 2.

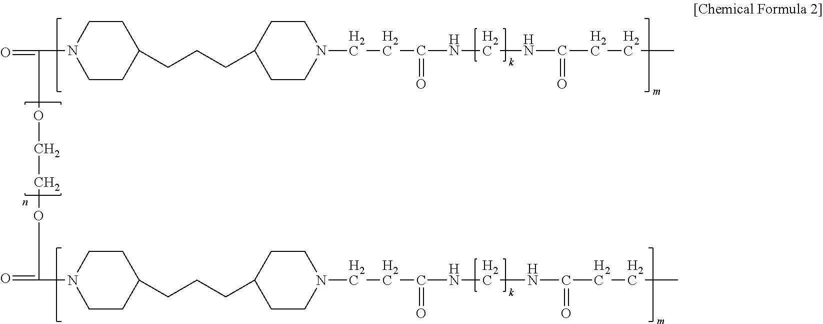

where k is an integer ranging from 4 to 10, n is an integer ranging from 11 to 45, and m is an integer ranging from 10 to 100.

Further, in the block copolymer, the hydrophilic PEG block may have a molecular weight of 1000 to 5000, a molecular weight ratio of. PEG and PAA may be 1:1 to 4, and its molecular weight may be 2,500 to 21,000.

Meanwhile, the pH-sensitive poly(β-aminoester urethane) (PAEU)-based block copolymer may be polyamino ester urethane-polyethylene glycol-polyamino ester urethane (PAEU-PEG-PAEU), PAEU-PCLA-PEG-PCLA-PAEU, PAEU-PCL-PEG-PCL-PAEU, PAEU-PCGA-PEG-PCGA-PAEU copolymer or (PCLA-PEG-PCLA-PAEU)x multicopolymer (wherein x is the number of repeating unit ranging from 1 to 20), or 4-arm PEG-PAEU copolymer.

Among the poly(β-aminoester urethane) (PAEU)-based block copolymers, the (PEG-PEAU)x multiblock copolymer may be a block copolymer represented by the following Chemical Formula 3.

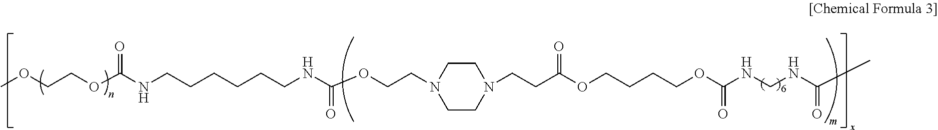

where n and m are independently the number of repeating unit ranging from 2 to 10, and x is the number of repeating unit ranging from 1 to 20.

In the multiblock copolymer, a number-average molecular weight (Mn) of the hydrophilic PEG polymer is not particularly limited, but preferably in the range of approximately 1,500 to 3,000. When the number-average molecular weight deviates from the above range, gel cannot be easily formed, and physical properties such as gel strength are decreased even if gel is formed, and thus it is difficult to apply it to a drug delivery system. The drug delivery system must be also injected without resistance upon hepatic arterial catheterization to be achieved. The preferred molecular weight ratio of the PEG and PAEU blocks is 1:1 to 1:3 and the preferred number-average molecular weight of the multiblock copolymer is 8,000 to 20,000. When they deviate from the above range, gel cannot be easily formed, or the gel viscosity is too high to perform injection through the guiding catheter.

Further, an example provides a drug delivery system, in which the polyaminourethaneurea(poly(β-aminourethaneurea), PAUU)-based block copolymer is polyaminourethaneurea-polyethyleneglycol-polyaminourethaneurea (PAUU-PEG-PAUU), PAUU-PCLA-PEG-PCLA-PAUU, PAUU-PCL-PEG-PCL-PAUU, PAUU-PCGA-PEG-PCGA-PAUU block copolymer, (PCLA-PEG-PCLA-PAUU)x multiblock copolymer, (PEG-PAUU)x multiblock copolymer (wherein x is the number of repeating unit ranging from 1 to 20), or 4-arm PEG-PAUU block copolymer.

Among the polyaminourethaneurea(poly(β-aminourethaneurea), PAUU)-based block copolymer, the (PEG-PAUU)x multiblock copolymer may be a block copolymer represented by the following Chemical Formula 4.

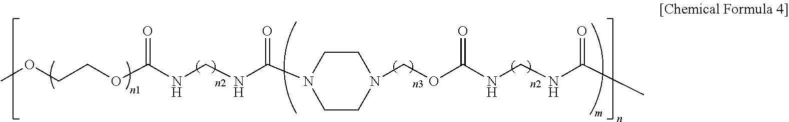

where n1 is an integer ranging from 7 to 50, n2 is an integer ranging from 2 to 8, n3 is an integer ranging from 1 to 10, and m is an integer ranging from 2 to 6.

In the multiblock copolymer, a number-average molecular weight (Mn) of the hydrophilic PEG polymer is not particularly limited, but preferably in the range of approximately 1,000 to 5,000. When the number-average molecular weight deviates from the above range, gel cannot be easily formed, and physical properties such as gel strength are decreased even if gel is formed, and thus it is difficult to apply it to a drug delivery system. The drug delivery system must be also injected without resistance upon hepatic arterial catheterization to be achieved. The preferred molecular weight ratio of the PAUU and PEG blocks is 1:1 to 1:3 and the preferred number-average molecular weight of the multiblock copolymer is 15,000 to 25,000. When they deviate from the above range, gel cannot be easily formed, or the gel viscosity is too high to perform injection through the guiding catheter.

Meanwhile, the pH-sensitive sulfonamide (SAM)-based block copolymer may be a sulfonamide-polyethylene glycol-sulfonamide (SAM-PEG-SAM), SAM-PCLA-PEG-PCLA-SAM, SAM-PCL-PEG-PCL-SAM or SAM-PCGA-PEG-PCGA-SAM copolymer.

Among the pH-sensitive sulfonamide-based block copolymers, the SAM-PCLA-PEG-PCLA-SAM block copolymer may be a block copolymer represented by the following Chemical Formula 5.

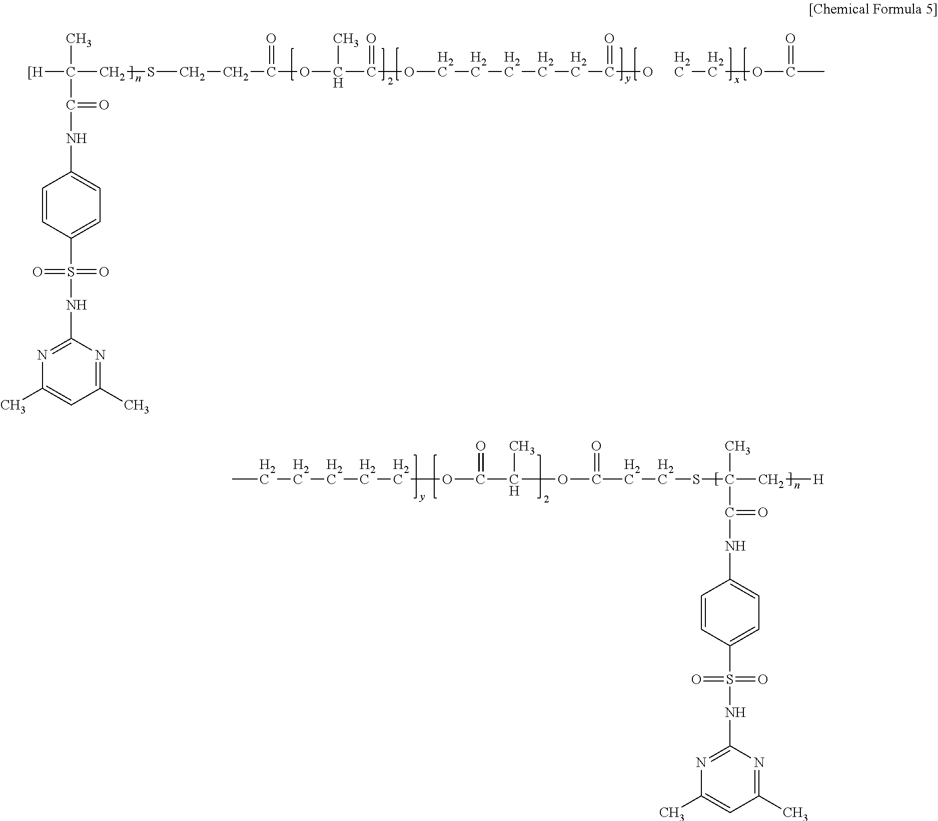

where x is an integer ranging from 10 to 50, y and z are independently an integer ranging from 0 to 50, and z and n are independently the number of repeating unit ranging from 1 to 100.

In the block copolymer, the molecular weight of PEG is 1500 to 2000, the molecular weight ratio of the hydrophobic and hydrophilic blocks is 1:1.2 to 1:1.8, the molar ratio of the hydrophobic PCL and PLA blocks is 2:1 to 3:1, and the number-average molecular weight of the block copolymer is 10,000 to 20,000.

Meanwhile, non-limiting examples of anticancer drugs as the applicable therapeutic drug may include doxorubicin (DOX), paclitaxel (PTX), 5-FU, cisplatin, camptothecin, docetaxel, Tamoxifen, anasterozole, carboplatin, topotecan, belotecan, irinotecan, gleevec and vincristine.

In an example, the drug delivery system may further include a contrast agent together with the therapeutic drug, in addition to the copolymer as the drug carrier. A non-limiting example of the contrast agent may include Lipiodol. When Lipiodol is used, the content may be 5 to 20 volume%, based on the 100 volume% of the copolymer. The content is preferable, considering appropriateness of x-ray angiography and easiness of catheter injection.

As for the drug delivery mechanism of the drug delivery system, the drug delivery system applicable to guiding catheterization of hepatic arterial embolization is the temperature and pH-sensitive block copolymer, and its preferred structure is a polymer hydrogel loaded with therapeutic drugs. In detail, a negatively or positively charged therapeutic drug is encapsulated in the temperature and pH-sensitive block copolymer hydrogel that is positively or negatively charged depending on pH of therapeutic environment. Ionization of pH-sensitive component of the polymer hydrogel is induced to be positively or negatively charged under the body conditions of pH 7.0 to 7.4 and temperature 37° C. and then, it is allowed to form an ionic complex or hydrogen bond with the negatively or positively charged therapeutic drug. Thus, the hydrogel in a sol-state is more stably loaded with the therapeutic drug and is injectable into the deeper microvessel in the tumor tissue. Once injected, the temperature and pH-sensitive copolymer loaded with the anticancer drug undergoes a transition from the sol into gel due to low pH of tumor tissue, and therefore it blocks blood supply from the artery to reduce the size and growth rate of the tumor, and also slowly releases the anticancer drug at a low concentration to maximize the therapeutic effects on liver cancer with minimal target organ/systemic toxicity. Accordingly, it can be used as an innovative therapeutic agent for advanced liver cancer.

FIG. 3 is a graph showing time-dependent DOX release of drug delivery systems of the following Examples 16 to 18. In the time-release curve, the slope at any point on the curve may be defined as a sustained release coefficient (sustained release rate), and the slope of the curve at the point of origin may be defined as the initial release coefficient. In addition, an average sustained release coefficient may be defined by the slope between the point of origin of the time-release curve and release curve at a particular time.

In this regard, those prepared by Examples have the initial release coefficient of 0.5 to 0, the 14-day average release rate of 5.6 to 6.7, and the 7-day average release rate of 6 to 6.45. These average release rates indicate that the drug delivery system was slowly degraded without rapid biodegradation behavior, and thus initial burst release of the therapeutic drug loaded on the drug delivery system did not occur. The DOX release was decreased over time, but the reduction was slow. Thus, the release was maintained for the desired period of time.

Hereinafter, Examples and Experimental Examples will be described. However, these Examples are for illustrative purposes only, and the invention is not intended to be limited by these Examples.

### EXAMPLES 1-4, COMPARATIVE EXAMPLE 1

### Preparation of Carriers for Hepatic Arterial Catheterization and Test on Easiness of Injection Via Guiding Catheter and In Vivo Clogging

### Example 1

A temperature and pH-sensitive, poly(amino ester urethane)-based block copolymer, (PEG-PAEU)x (x is a repeating unit of 1.89, number-average molecular weight is 8,520 g/mol) multiblock copolymer powder and PBS buffer were used to prepare a 20 wt % solution. 0.2 mL of Lipiodol was homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and easiness of injection via a guiding catheter and in vivo clogging were tested. As a result, injection was easily performed at a flow rate of 1 mL/min without clogging.

### Example 2

A temperature and pH-sensitive, polyaminourethaneurea-based block copolymer, (PEG-PAUU)x (x is a repeating unit of 2.79, number-average molecular weight is 19,500 g/mol) multiblock copolymer powder and PBS buffer were used to prepare a 15 wt % solution. 0.2 mL of Lipiodol was homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and easiness of injection via a guiding catheter and in vivo clogging were tested. As a result, injection was easily performed at a flow rate of 1 mL/min without clogging.

### Example 3

A temperature and pH-sensitive, poly(β-amino ester)-based block copolymer, PAE-PCLA-PEG-PCLA-PAE (each number-average molecular weight is 1300-1600-1600-1600-1300 g/mol) quintuple block copolymer powder and PBS buffer were used to prepare a 30 wt % solution. 0.2 mL of Lipiodol was homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and easiness of injection via a guiding catheter and in vivo clogging were tested. As a result, injection was easily performed at a flow rate of 1 mL/min without clogging.

### Example 4

A temperature and pH-sensitive, poly(amidoamine)-based block copolymer, PAA-PEG-PAA (each number-average molecular weight is 1580-4600-1580 g/mol) triple block copolymer powder and PBS buffer were used to prepare a 12.5 wt % solution. 0.2 mL of Lipiodol was homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and easiness of injection via a guiding catheter and in vivo clogging were tested. As a result, injection was easily performed at a flow rate of 1 mL/min without clogging.

### Example 5

A temperature and pH-sensitive, sulfonamide-based block copolymer, SAM-PCLA-PEG-PCLA-SAM (each number-average molecular weight is 6550-1650-1750-1650-6550 g/mol) quintuple block copolymer powder and PBS buffer were used to prepare a 20 wt % solution. 0.2 mL of Lipiodol was homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and easiness of injection via a guiding catheter and in vivo clogging were tested. As a result, injection was easily performed at a flow rate of 1 mL/min without clogging.

Examples 6-10: Preparation of Anticancer Drug-Encapsulated Carriers for Hepatic Arterial Catheterization and Test on Formulation Stability and In Vivo DOX Release

### Example 6

A temperature and pH-sensitive, poly(amino ester urethane)-based block copolymer, (PEG-PAEU)x (x is a repeating unit of 1.89, number-average molecular weight is 8,520 g/mol) multiblock copolymer powder and PBS buffer were used to prepare a 20 wt % solution. 6 mg of doxorubicin (DOX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and it showed good formulation stability.

### Example 7

A temperature and pH-sensitive, polyaminourethaneurea-based block copolymer, (PEG-PAUU)x (x is a repeating unit of 2.79, number-average molecular weight is 19,500 g/mol) multiblock copolymer powder and PBS buffer were used to prepare a 15 wt % solution. 6 mg of doxorubicin (DOX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and it showed good formulation stability.

### Example 8

A temperature and pH-sensitive, poly(β-amino ester)-based block copolymer, PAE-PCLA-PEG-PCLA-PAE (each number-average molecular weight is 1300-1600-1600-1600-1300 g/mol) quintuple block copolymer powder and PBS buffer were used to prepare a 30 wt % solution. 6 mg of doxorubicin (DOX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and it showed good formulation stability.

### Example 9

A temperature and pH-sensitive, poly(amidoamine)-based block copolymer, PAA-PEG-PAA (each number-average molecular weight is 1580-4600-1580 g/mol) triple block copolymer powder and PBS buffer were used to prepare a 12.5 wt % solution. 6 mg of doxorubicin (DOX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and it showed good formulation stability.

### Example 10

A temperature and pH-sensitive, sulfonamide-based block copolymer, SAM-PCLA-PEG-PCLA-SAM (each number-average molecular weight is 6550-1650-1750-1650-6550 g/mol) quintuple block copolymer powder and PBS buffer were used to prepare a 20 wt % solution. 6 mg of doxorubicin (DOX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and it showed good formulation stability.

### EXPERIMENTAL EXAMPLES 1 AND 2, EXAMPLES 11-15

### Transcatheter Arterial Chemoembolization of Hepatoma Animal Model with Anticancer Drug-Encapsulated Carriers for Hepatic Arterial Catheterization

### Experimental Example 1

### Preparation of Hepatoma Animal Model

Rabbit VX2 tumor provided by Deutsches Krebsforschungszentrum Tumorbank (Germany) was transplanted into the thigh of rabbits (New Zealand White, 2.5-3.0 kg). After 2 weeks, the rabbits having 15 mm-30 mm tumors were sacrificed by intravenous injection of 10 mL of pentothal sodium solution (62.5 mg/kg). The tumors were excised along with the tissues around them, after disinfection with iodine solution and alcohol, removing the hair, and cutting the skin over the tumor site. The tumor was cut to remove the central necrotic portion. The viable peripheral tumor tissue was mixed with calcium and magnesium-free Hank's balanced salt solution, and cut into very small pieces with scissors and scalpel until a smooth consistency was obtained. The tumor solution was mixed with 5 mL of RMPI-1640 (Rosewell Park Memorial Institute, Rosewell Park, N.Y.). The mixture was diluted to 1×10⁶ tumor cells/mL.

Injection and transplantation of tumors cell solution into the rabbit liver were performed as follows: firstly, a rabbit was anesthetized by intramuscular injection of 5 mg/kg of Zoletil, and 500 mL of phosphate buffered saline was administered through the ear vein via a 23 G needle. 500 mg of pentothal sodium was diluted in 40 mL of phosphate buffered saline, and this mixture (1.5 mL/kg) was injected through the rabbit ear vein at a flow rate of 1 mL/min. The hair in the abdomen was removed, and the skin was disinfected with iodine solution and alcohol to open the abdomen. 0.1 mL of the tumor tissue solution was injected to the liver parenchyma of the left lobe using an 18 G needle. The tumor tissue solution was injected to the left lobe among the 5 lobes in the rabbit liver since it is the easiest to observe with the ultrasound. After the injection of the tumor tissue solution, the rabbits were grown in a rabbit cage with normal meals. Within two weeks after the transplantation of tumor cells, a tumor was identified by ultrasound observation and CT. The tumor growth could be roughly predicted by the growth curve. Within three weeks after the transplantation of tumor cells, CT was performed to follow up the position and size of the tumor.

### Experimental Example 2

### Hepatic Artery Guiding Catheterization

After animals were anesthetized in the same manner as above, an 18G intravenous catheter was inserted into the ear artery, and selective cannulation of the left hepatic artery branches was performed using a 2.0-F microcatheter (Terumo, Tokyo, Japan) and a 0.018 inch micro-guide wire. At this time, 0.1 mg of prostaglandin was injected to prevent hepatic artery vasospasm after cannulation. Under X-ray angiography, the copolymer/doxorubicin/Lipiodol formulations prepared in Examples 13 to 16 were injected until the microvessels of the tumor were filled with the formulations.

### Example 11

The homogeneous copolymer/doxorubicin/Lipiodol formulation prepared in Example 6 was sterilized by passing through a syringe filter (Acrodisc; Pall Ann Arbor, Mich, 200 m pore size, PVDF filter). Under X-ray angiography, 0.7 mL of the sterilized copolymer/doxorubicin/Lipiodol formulation was injected to rabbit VX2 hepatoma model prepared in Experimental Example 1 using a hepatic artery guiding catheter until the microvessels of tumor were filled with the formulation. At this time, only 0.7 mL of Lipiodol was injected to the hepatoma model, and used as a control group for comparison. At 1 week after operation, the result of CT scan showed selective localization of the formulation in the tumor, as shown in FIG. 2.

### Example 12

The homogeneous copolymer/doxorubicin/Lipiodol formulation prepared in Example 7 was sterilized by passing through a syringe filter (Acrodisc; Pall Ann Arbor, Mich, 200 m pore size, PVDF filter). Under X-ray angiography, 0.7 mL of the sterilized copolymer/doxorubicin/Lipiodol formulation was injected to rabbit VX2 hepatoma model prepared in Experimental Example 1 using a hepatic artery guiding catheter until the microvessels of tumor were filled with the formulation. At this time, only 0.7 mL of Lipiodol was injected to the hepatoma model, and used as a control group for comparison.

### Example 13

The homogeneous copolymer/doxorubicin/Lipiodol formulation prepared in Example 8 was sterilized by passing through a syringe filter (Acrodisc; Pall Ann Arbor, Mich, 200 m pore size, PVDF filter). Under X-ray angiography, 0.7 mL of the sterilized copolymer/doxorubicin/Lipiodol formulation was injected to rabbit VX2 hepatoma model prepared in Experimental Example 1 using a hepatic artery guiding catheter until the microvessels of tumor were filled with the formulation. At this time, only 0.7 mL of Lipiodol was injected to the hepatoma model, and used as a control group for comparison.

### Example 14

The homogeneous copolymer/doxorubicin/Lipiodol formulation prepared in Example 9 was sterilized by passing through a syringe filter (Acrodisc; Pall Ann Arbor, Mich, 200 m pore size, PVDF filter). Under X-ray angiography, 0.7 mL of the sterilized copolymer/doxorubicin/Lipiodol formulation was injected to rabbit VX2 hepatoma model prepared in Experimental Example 1 using a hepatic artery guiding catheter until the microvessels of tumor were filled with the formulation. At this time, only 0.7 mL of Lipiodol was injected to the hepatoma model, and used as a control group for comparison.

### Example 15

The homogeneous copolymer/doxorubicin/Lipiodol formulation prepared in Example 10 was sterilized by passing through a syringe filter (Acrodisc; Pall Ann Arbor, Mich, 200 m pore size, PVDF filter). Under X-ray angiography, 0.7 mL of the sterilized copolymer/doxorubicin/Lipiodol formulation was injected to rabbit VX2 hepatoma model prepared in Experimental Example 1 using a hepatic artery guiding catheter until the microvessels of tumor were filled with the formulation. At this time, 0.7 mL of Lipiodol was only injected to the hepatoma model, and used as a control group for comparison.

### EXAMPLE 16-20

### Analysis of Doxorubicin Concentration in hepatoma after transcatheter arterial chemoembolization with copolymer/doxorubicin/Lipiodol composition

### Example 16

At one week after transcatheter arterial chemoembolization, the hepatoma models of Example 11 were sacrificed and the livers were taken out. The doxorubicin concentration was determined by HPLC in the liver tissues which were divided into three groups: where the copolymer/doxorubicin/Lipiodol formulation was visually identified, the copolymer/doxorubicin/Lipiodol formulation was not visually identified, and the normal liver tissue neighboring the tumor.

### Example 17

At one week after hepatic arterial embolization, the hepatoma models of Example 12 were sacrificed and the livers were taken out. The doxorubicin concentration was determined by HPLC in the liver tissues which were divided into three groups: where the copolymer/doxorubicin/Lipiodol formulation was visually identified, the copolymer/doxorubicin/Lipiodol formulation was not visually identified, and the normal liver tissue neighboring the tumor.

### Example 18

At one week after hepatic arterial embolization, the hepatoma models of Example 13 were sacrificed and the livers were taken out. The doxorubicin concentration was determined by HPLC in the liver tissues which were divided into three groups: where the copolymer/doxorubicin/Lipiodol formulation was visually identified, the copolymer/doxorubicin/Lipiodol formulation was not visually identified, and the normal liver tissue neighboring the tumor.

### Example 19

At one week after hepatic arterial embolization, the hepatoma models of Example 14 were sacrificed and the livers were taken out. The DOX concentration was determined by

HPLC in the liver tissues which were divided into three groups: where the copolymer/doxorubicin/Lipiodol formulation was visually identified, the copolymer/doxorubicin/Lipiodol formulation was not visually identified, and the normal liver tissue neighboring the tumor.

### Example 20

At one week after hepatic arterial embolization, the hepatoma models of Example 15 were sacrificed and the livers were taken out. The DOX concentration was determined by HPLC in the liver tissues which were divided into three groups: where the copolymer/doxorubicin/Lipiodol formulation was visually identified, the copolymer/doxorubicin/Lipiodol formulation was not visually identified, and the normal liver tissue neighboring the tumor.

The results of Examples 16 to 20 are shown in FIG. 3, in which it is compared with the hepatoma model without hepatic arterial embolization as a control group.

### Comparative Example 1

A temperature and pH-sensitive (PEG-PAEU)x (x is a repeating unit of 1.89, number-average molecular weight is 8,520 g/mol) multiblock copolymer powder and PBS buffer were used to prepare a 20 wt % solution. 0.7 mL of the copolymer solution was prepared as a carrier for hepatic arterial catheterization without Lipiodol, and sterilized by passing through a syringe filter (Acrodisc; Pall Ann Arbor, Mich, 200 m pore size, PVDF filter). Finally, it was tested whether the formulation was applicable to X-ray angiography. As a result, because Lipiodol was not contained, and thus there is no iodine, the angiography is difficult.

### Comparative Example 2

A temperature and pH-sensitive, poly(amino ester urethane)-based block copolymer, (PEG-PAEU)x (x is a repeating unit of 1.89, number-average molecular weight is 8,520 g/mol) multiblock copolymer powder and PBS buffer were used to prepare a 20 wt % solution. 1 mg of doxorubicin (DOX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a carrier for angiographic hepatic arterial catheterization was prepared, and it showed good formulation stability.

### Comparative Example 3

A temperature and pH-sensitive, poly(amino ester urethane)-based block copolymer, (PEG-PAEU)x (x is a repeating unit of 1.89, number-average molecular weight is 8,520 g/mol) multiblock copolymer powder and PBS buffer were used to prepare a 20 wt % solution. 10 mg of DOX and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a high-dose DOX-encapsulated carrier for hepatic arterial catheterization was prepared, and it showed good formulation stability.

### Comparative Example 4

A temperature and pH-sensitive, poly(amino ester urethane)-based block copolymer, (PEG-PAEU)x (x is a repeating unit of 1.89, number-average molecular weight is 8,520 g/mol) multiblock copolymer powder and PBS buffer were used to prepare a 20 wt % solution. 6 mg of poorly-water soluble paclitaxel (PTX) and 0.2 mL of Lipiodol were homogeneously mixed with 0.8 mL of the copolymer solution at room temperature, and sterilized by passing through a syringe filter (Acrodisc; Pall Ann Arbor, Mich, 200 m pore size, PVDF filter). Finally, a therapeutic PTX-encapsulated carrier for hepatic arterial catheterization was prepared, and compared with the DOX formulation. As a result, the formulation could be easily prepared without additional solubilizing agents.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A drug delivery system for the treatment of liver cancer that is based on interventional injection of a temperature and pH-sensitive hydrogel,
wherein the drug delivery system is composed of a block copolymer applicable to hepatic arterial catheterization and a therapeutic agent is loaded inside the drug delivery system, and
wherein the drug delivery system is in the sol state outside, and undergoes a phase transition into the gel state inside the hepatic artery, thereby delaying or blocking blood supply of the hepatic artery, and slowly releasing the therapeutic agent during the phase transition into the gel state inside the hepatic artery.

2. The drug delivery system according to claim 1, wherein a contrast agent is further included inside the drug delivery system.

3. The drug delivery system according to claim 1, wherein the block copolymer is a temperature and pH-sensitive poly(β-aminoester)-based block copolymer, a poly(amidoamine)-based block copolymer, a poly(aminoesterurethane)-based block copolymer, a poly(aminourethaneurea)-based block copolymer, or a sulfonamide-based block copolymer.

4. The drug delivery system according to claim 1, wherein a hydrophilic block component of the block copolymer is polyethylene glycol.

5. The drug delivery system according to claim 1, wherein a hydrophobic and biodegradable block component of the block copolymer is one or more selected from the group consisting of polylactic acid (PLA), polyglycolic acid (PGA), polycaprolactone (PCL), poly(caprolactone-lactide) random copolymer (PCLA), poly(caprolactone-glycolide) random copolymer (PCGA), and poly(lactide-glycolide) random copolymer (PLGA).

6. The drug delivery system according to claim 3, wherein the poly(β-amino ester)(PAE)-based block copolymer is poly(β-amino ester)-polyethylene glycol-poly(β-amino ester) (PAE-PEG-PAE), PAE-PCLA-PEG-PCLA-PAE, PAE-PCL-PEG-PCL-PAE, or PAE-PCGA-PEG-PCGA-PAE.

7. The drug delivery system according to claim 6, wherein the poly(β-amino ester)-based block copolymer is defined by the following Chemical Formula: 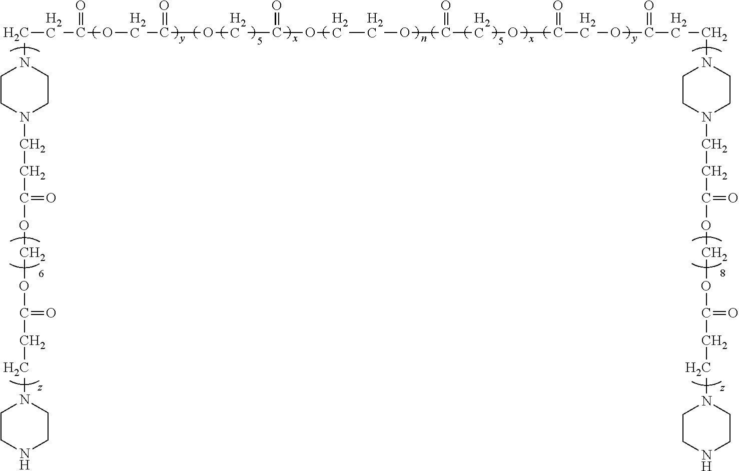
wherein x and y are independently an integer ranging from 0 to 50, and z and n are independently an integer ranging from 1 to 100.

8. The drug delivery system according to claim 3, wherein the poly(amidoamine)(PAA)-based block copolymer is a poly(amidoamine)-polyethylene glycol-poly(amidoamine) (PAA-PEG-PAA), PAA-PCLA-PEG-PCLA-PAA, PAA-PCL-PEG-PCL-PAA, or PAA-PCGA-PEG-PCGA-PAA copolymer.

9. The drug delivery system according to claim 8, wherein the poly(amidoamine)-based block copolymer is defined by the following Chemical Formula: 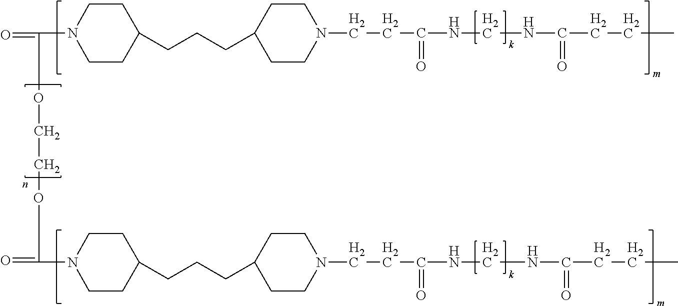
wherein k is an integer ranging from 4 to 10, n is an integer ranging from 11 to 45, and m is an integer ranging from 10 to 100.

10. The drug delivery system according to claim 3, wherein the poly(aminoester urethane) (PAEU)-based block copolymer is polyamino ester urethane-polyethylene glycol-polyamino ester urethane (PAEU-PEG-PAEU), PAEU-PCLA-PEG-PCLA-PAEU, PAEU-PCL-PEG-PCL-PAEU, PAEU-PCGA-PEG-PCGA-PAEU copolymer or (PCLA-PEG-PCLA-PAEU)x multicopolymer (wherein x is the number of repeating unit ranging from 1 to 20), 4-arm PEG-PAEU copolymer.

11. The drug delivery system according to claim 10, wherein the poly(amino ester urethane)-based block copolymer is defined by the following Chemical Formula: 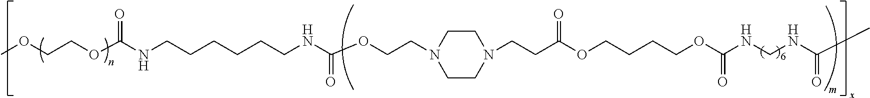
wherein n and m are independently the number of repeating unit ranging from 2 to 10, and x is the number of repeating unit ranging from 1 to 20.

12. The drug delivery system according to claim 3, wherein the poly(aminourethaneurea) (poly(β-aminourethaneurea), PAUU)-based block copolymer is polyaminourethaneurea-polyethyleneglycol-polyaminourethaneurea (PAUU-PEG-PAUU), PAUU-PCLA-PEG-PCLA-PAUU, PAUU-PCL-PEG-PCL-PAUU, PAUU-PCGA-PEG-PCGA-PAUU block copolymer, (PCLA-PEG-PCLA-PAUU)x multiblock copolymer, (PEG-PAUU)x multiblock copolymer (wherein x is the number of repeating unit ranging from 1 to 20), or 4-arm PEG-PAUU block copolymer.

13. The drug delivery system according to claim 12, wherein the poly(aminourethaneurea)-based multiblock copolymer is defined by the following Chemical Formula: 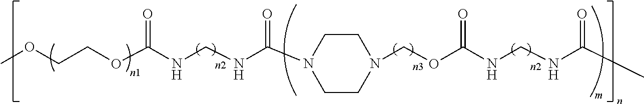
wherein n1 is an integer ranging from 7 to 50, n2 is an integer ranging from 2 to 8, n3 is an integer ranging from 1 to 10, and m is an integer ranging from 2 to 6.

14. The drug delivery system according to claim 3, wherein the sulfonamide(SAM)-based block copolymer is a sulfonamide-polyethylene glycol-sulfonamide (SAM-PEG-SAM), SAM-PCLA-PEG-PCLA-SAM, SAM-PCL-PEG-PCL-SAM or SAM-PCGA-PEG-PCGA-SAM copolymer.

15. The drug delivery system according to claim 14, wherein the sulfonamide-based block copolymer is defined by the following Chemical Formula: 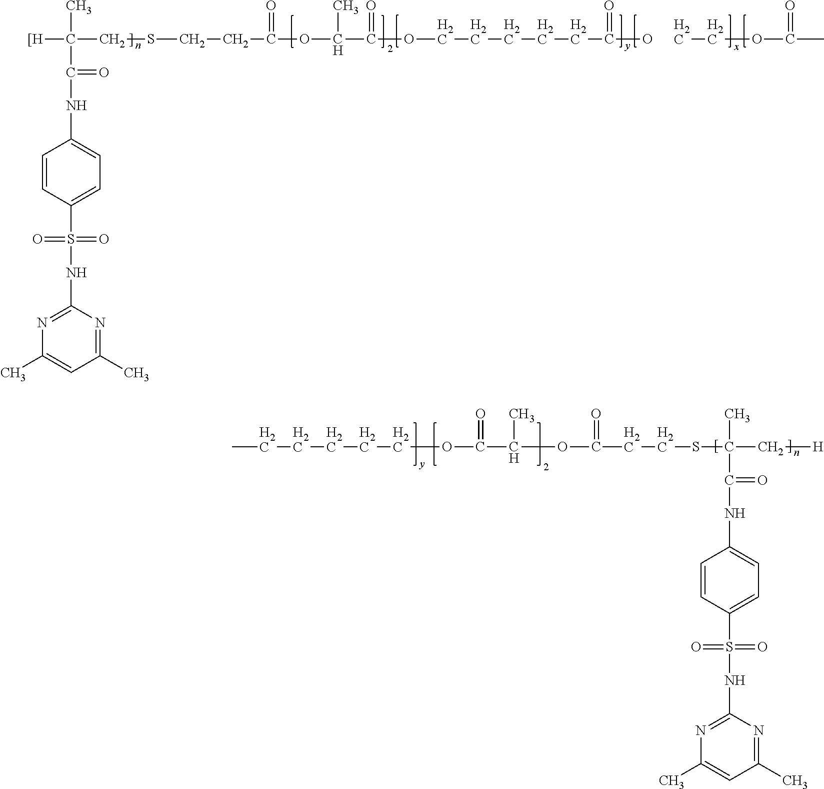
wherein x is an integer ranging from 10 to 50, y and z are independently an integer ranging from 0 to 50, and z and n are independently the number of repeating unit ranging from 1 to 100.

16. The drug delivery system according to claim 2, wherein the contrast agent is included in a volume of 5 to 20 volume %, based on the volume of the copolymer.

17. The drug delivery system according to claim 1, wherein an initial release coefficient is 0.5 to 0 when the initial release coefficient is defined by the slope of the curve at the point of origin in the time-release curve of the drug delivery system.
